(19) United States
(12) Patent Application Publication
(10) Pub. No.: US 2007/0218105 A1
(43) Pub. Date: Sep. 20, 2007

(54) Topical Treatment with NGF and DHA in Damaged Corneas

(76) Inventors: Salomon Esquenazi, Aventura, FL (US); Haydee E. Bazan, New Orleans, LA (US); Nicolas G. Bazan, New Orleans, FL (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/577,239

(22) PCT Filed: Sep. 16, 2005
(86) PCT No.: PCT/US05/33386

§ 371 Date: Apr. 27, 2007

Related U.S. Application Data
(60) Provisional application No. 60/620,459, filed on Oct. 19, 2004.

(51) Int. Cl. A61K 38/18; A61F 9/00

(52) U.S. Cl. 424/428; 514/2

(57) ABSTRACT

The topical administration of a combination of nerve growth factor (NGF) and docosahexaenoic acid (DHA) has been discovered to synergistically increase the effects of NGF in re-innervating the cornea. This enhancement in corneal nerve re-growth will yield a faster anatomical and functional recovery after PRK or LASIK surgeries. Using rabbits, the application of NGF and DHA resulted in increased corneal nerve surface area, increased epithelial proliferation, and decreased rose bengal staining as compared with NGF, DHA, or vehicle control individually. The topical application of NGF plus DHA in accelerating the re-innervation after PRK or LASIK, will help avoid or alleviate the symptoms of dry eye or other neurotrophic keratopathies due to corneal injuries. The topical application can be by using a corneal shield or lens. This treatment will also be useful in other corneal abnormalities including those caused by chemical burn, congenital corneal neuropathy, or acquired corneal neuropathy.

The benefit of the filing date of provisional application 60/620,459 filed 19 Oct. 2004 is claimed under 35 U.S.C. § 119(e) in the United States, and is claimed under applicable treaties and conventions in all countries

The development of this invention was partially funded by the United States Government under grants R01EY04928, R01EY06635, and R01EY05121 from the United States Public Health Service, and grant P30EY02377 from the National Institutes of Health. The United States Government has certain rights in this invention.

### TECHNICAL FIELD

This invertion pertains to a new composition and new method to enhance corneal nerve re-growth after injury to the cornea either by trauma or surgery (e.g., PRK or LASIK) by topically administering a combination of nerve growth factor (NGF) and docosahexaenoic acid (DHA).

### BACKGROUND ART

The use of the excimer laser for the correction of refractive defects is widely accepted today. An annual survey that assesses the variety and volume of refractive surgeries showed that excimer ablative refractive procedures are the predominant type performed since 1998. See D. V. Leaming, “Practice styles and preferences of ASCRS member-2003 survey,” J. Cataract Refract. Surg., vol. 30, pp. 892-900 (2004). Photorefractive keratectomy (PRK) consists of the removal of the epithelium before applying the laser correction. On the other hand, laser in situ keratomileusis (LASIK) requires the creation of a flap that includes epithelium and superficial stroma before the laser treatment. Although the cornea is virtually avascular, it is densely innervated, and in both procedures, damage to the corneal nerve supply occurs. This damage results in a neurotrophic epitheliopathy and dry eye symptoms, characterized by punctuate epithelial erosions occurring days to weeks after the refractive procedure. In LASIK, the hinge position and flap thickness seem to be important factors contributing to the rate of corneal sensation compromise. See B. A. Nassaralla et al., “The effect of hinge position and depth plate on the rate of recovery of corneal sensation following LASIK,” Am. J. Ophthalmol., vol. 139, pp. 118-124 (2005). An impaired corneal sensitivity causes a reduction in afferent input and a loss of the lacrimal reflex with a subsequent decrease in essential tear-derived trophic factors. See S. E. Wilson, “Laser in situ keratomileusis-induced (presumed) neurotrophic epitheliopathy,” Ophthalmology. Vol. 108, pp. 1082-1087 (2001); and C. Belmonte et al., “Neural basis of sensation in intact and injured corneas,” Exp. Eye Res., vol. 78, pp. 513-525 (2004). However, no correlation was found between a decrease in tear production as measured by Schirmer's test and a change in corneal sensitivity in human patients that underwent LASIK surgery. See A. Michaeli et al., “The effects of Laser in situ keratomileusis on tear secretion and corneal sensitivity,” J. Refract. Surg., vol. 20, pp. 379-383 (2004). Tears provide not only lubrication, but also deliver growth factors and proteins to the compromised ocular surface that are essential for the maintenance of epithelial integrity following corneal refractive surgery. In addition, chronic dry eyes are associated with an enhanced regression of the PRK correction. See S. Esquenazi, “Five year follow-up of laser in situ keratomileusis for hyperopia using the keracor 117C excimer laser,” J. Refract. Surg., vol. 20, pp. 356-363 (2004). Additionally, the local production of neuronal-derived molecules from sub-basal and epithelial nerve bundles may promote a healthy epithelium. If the corneal nerve bed remains compromised, evidence suggests that the homeostasis of the cornea is disrupted resulting in impaired healing and persistent epithelial erosions. See T. W. Mittag et al., “Trophic functions of the neuron, V: familial dysautonomia: choline acetytransferase in familial dysautonomia,” Ann. N.Y. Acad. Sci., vol. 228, pp. 301-306 (1974); and V. Puangsricharern et a., “Cytologic evidence of corneal diseases with limbal stem cell deficiency,” Ophthalmology, vol. 102, pp. 1476-1485 (1995). Studies have shown that even three years after LASIK and PRK surgery, the number of corneal nerves has not returned to the preoperative densities. See M. P. Calvillo et al., “Corneal reinnervation after LASIK: Prospective 3-year longitudinal study,” Invest. Ophthalmol. Vis. Sci., vol. 45, pp. 3991-3996 (2004); and J. C. Erie, “Corneal wound healing after photorefractive keratectomy: a 3-year confocal microscopy study,” Trans. Am. Ophthalbnol. Soc., vol. 101, pp. 293-333 (2003). Therefore, facilitating corneal reinnervation following either PRK or LASIK is important to restore normal, physiologic functions of the cornea.

Current evidence indicates that nerve growth factor (NGF), a neurotrophic and immunomodulatory mediator, is responsible for the growth, differentiation, and survival of sensory neurons and acceleration of wound healing. See R. Levi-Montalcini, “The nerve growth factor 35 years later,” Science, vol. 237, pp. 1154-1162 (1997); and S. S. Riaz et al., “Neurotrophic factors in peripheral neuropathies: pharmacological strategies,” Prog. Neurobiol., vol. 49, pp. 125-43 (1996). Keratocytes, epithelial cells, and endothelial cells synthesize NGF. Also, epithelial cells express NGF receptors. Following an injury, an upregulation of corneal NGF and its receptors has been shown. See A. Lambiase et al., “Nerve growth factor promotes corneal healing: structural, biochemical, and molecular analyses of rat and human comeas,” Invest. Ophthal. Vis. Sci., vol. 41, pp. 1063-1069 (2000); and L. You et al., “Neurotrophic factors in the human cornea,” Invest. Ophthalmol. Vis. Sci., vol. 41, pp. 692-702 (2000). Topically administered NGF was found to promote healing of refractory corneal neurotrophic ulcers. A role for NGF in modulating epithelial-stromal communication, which is important in the induction of stromal healing, has been postulated. See A. Lambiase et al., “Topical treatment with nerve growth factor for corneal neurotrophic ulcers,” N. Engl. J. Med., vol. 338, pp. 1174-1180 (1998); and S. Bonini et al., “Topical treatment with nerve growth factor for neurotrophic keratitis,” Ophthalmology, vol. 107, pp. 1347-1351 (2000). In addition, corneal sensitivity after LASIK has been enhanced by the administration of topical NGF. See M. J. Joo et al., “The effect of nerve growth factor on corneal sensitivity after laser in situ keratomileusis,” Arch. Ophthalmol., vol. 122, pp. 1338-1341 (2004). If the effect of NGF on corneal wound healing could be enhanced, the restoration of ocular surface integrity and visual function would be faster and more complete.

Besides NGF, other substances, such as substance P (SP) and calcitonin gene-related peptide (CGRP), have been postulated to help with corneal wound healing. (Belmonte et al., 2004). The topical application of autologous serum, which harbored various neurotrophic factors, was shown to promote healing in epithelial disorders in neurotrophic keratopathy. See Y. Matsumoto et al., “Autologous serum application in the treatment of neurotrophic keratopathy,” Ophthalmology, vol. 111, pp. 1115-1120 (2004). Presence of known neural healing factors (substance P, insulinlike growth factor, and nerve growth factor) was confirmed in the autologous serum.

The omega-3 fatty acid docosahexaenoic acid (22:6, n-3, DHA) is highly concentrated in synapses, is required during development and for synaptic plasticity, and participates in neuroprotection. DHA is most concentrated in photoreceptors and in brain and retinal synapses. In the cornea, DHA is a minor component of membrane phospholipids. DHA is also used continuously in the biogenesis and maintenance of neuronal and photoreceptor membranes. See N. G. Bazan, “Synaptic lipid signaling: significance of polyunsaturated fatty acids and platelet-activating factor,” J. Lipid Res., vol. 44, pp. 2221-2233 (2003); and H. E. P. Bazan et al., “Composition of phospholipids and free fatty acid and incorporation of labeled arachidonic acid in rabbit cornea. Comparison of epithelium, stroma and endothelium,” Curr. Eye Res., vol. 3, pp. 1313-1319 (1984). Free DHA is released through phospholipases from membrane phospholipids in response to seizures. See N. G. Bazan, “Effects of ischemia and electroconvulsive shock on free fatty acid pool in the brain,” Biochim. Biophys. Acta, vol. 218, pp. 1-10 (1970); and D. L. Birkle et al., “Effect of bicuculline-induced status epilepticus on prostaglandins and hydroxyeicosatetraenoic acids in rat brain subcellular fractions,” J. Neurochem., vol. 48, pp. 1768-1778 (1987). Recently the structure and bioactivity of neuroprotectin D1, a potent DHA-derived mediator in brain ischemia-reperfusion and in oxidative stress, has been described. See V. L. Marcheselli et al., “Novel docosanoids inhibit brain ischernia-reperfusion-mediated leukocyte infiltration and pro-inflammatory gene expression,” J. Biol. Chem., vol. 278, pp. 43807-817 (2003); and P. K. Mukherjee et al., “Neuroprotectin D1: A docosahexaenoic acid-derived docosatriene protects human retinal pigment epithelial cells from oxidative stress,” Proc. Natl. Acad. Sci., USA, vol. 101, pp. 8491-96 (2004). Docosahexaenoic acid (DHA) has also been used to slow the progression of X-linked Retinitis pigmentosa. See F. L. Berson et al., “Clinical trial of docosahexaenoic acid in patients with retinitis pigmentosa receiving vitamin A treatment,” Arch. Ophthalmol., vol. 122, pp. 1297-1314 (2004); and D. R. Hoffman et al., “A randomized, placebo-controlled clinical trial of docosahexaenoic acid supplementation for X-lined retinitis pigmentosa,” Am. J. Ophthalmol., vol. 137, pp. 704-718 (2004).

While there is increasing support that NGF promotes corneal wound healing, there exists an unfilled need for enhancing the effect of NGF on corneal nerve regeneration after trauma or corneal lamellar refractive surgery.

### DISCLOSURE OF INVENTION

We have discovered that the topical administration of a new combination of nerve growth factor (NGF) and docosahexaenoic acid (DHA) enhances the effects of NGF in re-innervating the cornea. This enhancement in corneal nerve re-growth will yield a faster anatomical and functional recovery after PRK or LASIK surgeries. Using rabbits, the application of NGF and DHA resulted in increased corneal nerve surface area, increased epithelial proliferation, and decreased rose bengal staining when compared with application of NGF, DHA, or albumin individually. The topical application of NGF plus DHA to accelerate corneal re-innervation after PRK or LASIK surgeries will help avoid or lessen the symptoms of dry eye or other neurotrophic keratopathies. This treatment will also be useful in other corneal abnormalities including those caused by chemical burn, congenital corneal neuropathy, or acquired corneal neuropathy.

### BRIEF DESCRIPTION OF DRAWINGS

FIG. 1 illustrates the results of staining corneal epithelium with monoclonal Ki-67 antibody, an indication of proliferative cells, in tissue from rabbit corneas 8 weeks after PRK in a control group and in three groups treated with NGF, DHA, or NGF plus DHA.

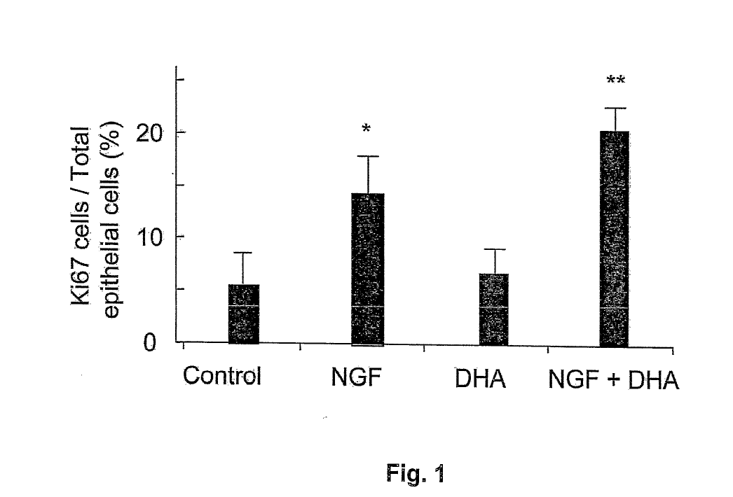

FIG. 2A illustrates the results of calculating the area of the sub-basal nerve bundles based upon tubulin staining in tissue from rabbit corneas 8 weeks after PRK in a control group and in three groups treated with NGF, DHA, or NGF plus DHA.

FIG. 2B illustrates the results of calculating the area of the epithelial nerve bundles based upon tubulin staining in tissue from rabbit corneas 8 weeks after PRK in a control group and in three groups treated with NGF, DHA, or NGF plus DHA.

FIG. 3A illustrates the ratio of nerve area to total tissue area of the sub-basal layer based upon CGRP-positive immunofluourescent staining in tissue from rabbit corneas 8 weeks after PRK in a control group and in three groups treated with NGF, DHA, or NGF plus DHA.

FIG. 3B illustrates the ratio of nerve area to total tissue area of the epithelial layer based upon CGRP-positive immunofluourescent staining in tissue from rabbit corneas 8 weeks after PRK in a control group and in three groups treated with NGF, DHA, or NGF plus DHA.

FIG. 4 illustrates the ratio of nerve area to total tissue area of the sub-basal layer based upon Substance P-positive staining in tissue from rabbit corneas 8 weeks after PRK in a control group and in three groups treated with NGF, DHA, or NGF plus DHA.

### MODES FOR CARRYING OUT THE INVENTION

### EXAMPLE 1

Materials and Methods

Photorefractive Surgery

Twenty-one New Zealand albino rabbits weighing 1.5 to 2.0 kg were obtained from a commercial supplier and were treated in accordance with the guidelines of the Association for Research in Vision and Ophthalmology. Each rabbit received intramuscular xylazine (10 mg/kg) and ketamine hydrochloride (50 mg/kg) anesthesia. Tetracaine eye drops were used as topical anesthesia. Tear secretion tests (e.g., tear break-up time, Schirmer's test, and rose bengal staining) were performed preoperatively under general anesthesia. Each rabbit received a unilateral PRK laser treatment. The corneal epithelium was removed with an epithelial scrubber (Katena Inc, Denville, N.J.). An excimer laser ablation to correct −5 diopters of myopia using a 6.5 mm optical zone (Lasersight Technologies, Inc., Winter Park, Fla.) was performed. The eye was washed with balanced salt solution. Lubricating eye drops and an ophthalmic solution of 0.3% ofloxacin eye drops (Allergan Inc, Irvine, Calif.) were used postoperatively. Unless otherwise indicated, all chemicals were purchased from Sigmna Chemical Co. (St. Louis, Mo.).

Preparation of NGF and DHA

NGF was purchased (Sigma Chemical Co.) and prepared in a stock solution of 6.0 μg in 1.5 ml PBS and kept at 4° C. DHA was also purchased and then complexed to 25% human albumin (Baxter Healthcare, Deerfield, Ill.) in a proportion of 1 ml albumin to 1 mg of DHA. The DHA-albumin complex was kept in the dark at 4° C. in a sterile bottle until use.

PRK and Treatment of Animals

Twenty-one New Zealand albino rabbits were divided in 4 groups. The three experimental groups consisted of 6 rabbits each. The control group had only 3 rabbits. The rabbits within each of the four groups were randomized to receive twice-weekly topical treatments through 72-hour collagen shields (Oasis Medical Inc.; Glendora, Calif.) for a total of eight weeks. The four treatments were as follows: (1) 0.1 μg NGF (25 μl) plus 100 μl phosphate buffered saline (PBS); (2) 100 μg DHA (100 μl) plus 25 μl of PBS; (3) 0.1 μg NGF (25 μl) plus 100 μg DHA (100 μl); and (4) 125 μl of PBS with albumin (control). In all animals, a tarsorrhaphy was performed on the treated eyes, and the eyes were opened only twice a week to introduce a new collagen shield.

Statistical Analysis

Statistical analyses were performed using the Statistical Analysis System (SAS) software version 9.0 (SAS Institute, Cary, N.C.). The tear secretion tests (Schirmer, tear break-up time and rose bengal staining) and density of nerve areas were analyzed using a repeated measures design in the analysis of variance (ANOVA). The differences in the tear secretion tests between the four treatment groups were analyzed at each time point. The effect of the various treatments on tubulin III-, CGRP- and SP-positive epithelial and sub-basal nerve areas was evaluated with a multivariate test. Comparisons among the four treatments groups were made using adjusted least square means with alpha levels corrected by a simulation method.

### EXAMPLE 2

Absorption of DHA-Albumin Solution by Collagen Shields

Measurements of the absorption of the DHA: albumin solution by the collagen shields were performed through tandom mass spectrometry analysis. Corneal shields were soaked with the DHA: albumin solution overnight. The shields were washed in PBS (pH 7.4), and then extracted in 1 ml 100% methanol, followed by a 1 ml methanol wash. Collected solvent extracts were dried under nitrogen and re-suspended in 1 ml methanol. The samples were then analyzed using a liquid chromatograph-tandem mass spectrometer (LC-MSMS; LC-TSQ Quantum, Thermo Electron Corp.; Waltham, Mass.), installed with a Biobasic-AX column (Thermo-Hypersil-Keystone) and eluted with 100% solution A (40:60:0.01 methanol/water/acetic acid; pH 4.5) to 100% solution B (99.99:0.01 methanol/acetic acid)], at a flow rate of 300 μl/min for 30 minutes. LC effluents were diverted to an electro spray-ionization probe (ESI) on a TSQ Quantum (Thermo Electron) triple quadrupole mass spectrometer. DHA standards (Cayman Chem.; Ann Arbor, Mich.) were used for optimizing the analysis and for creating calibration curves. The instrument was set on full-scan mode and selected reaction modes for quantitative analysis to detect parent ions and product ions simultaneously. The selected parent ion was 327.2 m/z, and the selected product ion was 283.3 m/z at a collision energy of 16 V, running on negative ion detection mode. Quantification of DHA was measured by integration of peak areas of samples and standards. (Data not shown)

Two Soft Shield, Collagen shields, 72-hour (Oasis Medical Inc., Glendora, Calif.), two hilafilcon B soft 2-week contact lenses (Bausch & Lomb, Rochester, N.Y.), and two Night and Day soft contact lenses (Ciba Vision, Duluth, Ga.) were tested to determine the absorption of DHA. After soaking the materials in the DHA: albumin as in Example 1, the lipids were extracted and analyzed by mass spectrometry. A peak with retention time of 20.6 minutes corresponding to DHA was observed with the samples. The 72-hour collagen shield absorbed DHA with more efficiency (25%) as compared with the hilafilcon B soft 2-week contact lens (17.6%) and the Night and Day contacts (15%). Thus the 72-hour collagen shields contain more DHA to be delivered to the cornea and were used in the remaining experiments.

### EXAMPLE 3

Tear Secretion Tests

Tear secretion tests (tear break-up time, Schirmer's test and rose bengal staining) were performed every 15 days over the 8-week period following PRK. All the tests were performed with general anesthesia. Schirmer's test was performed using the recommended method with the Alcon test strips (Alcon Laboratories; Forth Worth, Tex.). The tear break-up time test was performed using fluorescein strips (Akorn Inc.; Lincolnshire, Ill.) that were moistened with non-preserved saline solution. Rose bengal staining was performed with Barnes/Hind strips (Akorn, Inc.). Three or mnore punctate spots of staining on the cornea were required to consider the stain positive. All measurements were conducted in a blinded fashion.

The animals tolerated the treatments well, and no adverse reactions were noted throughout the length of the experiment. No significant difference was found in tear secretion, as measured by Schirmer's test, at any time point (Table 1). Fifteen days after surgery, the average Schirmer values were 10.5 mm, 11.5 mm, 10 mm, and 12 mm for control, NGF-, DHA-, and NGF plus DHA-treated groups, respectively (P=0.58). At 1 month, the values were 11 mm, 12 mm, 11.5 mm, and 12.5 mm for control, NGF-, DHA- and NGF plus DHA-treated groups, respectively (P=0.62).

TABLE 1
Tear Secretion Tests After PRK in Rabbit Corneas*

| | Schirmer's Test (mm) | | | Tear Break-Up Time (sec) | | |
|---|---|---|---|---|---|---|
| Treatment | 15 Days | 30 Days | 45 Days | 15 Days | 30 Days | 45 Days |
| Control | 10.5 ± 3.8 | 11.0 ± 4.2 | 12.0 ± 3.2 | 13.0 ± 4.4 | 12.5 ± 5.2 | 12.5 ± 4.2 |
| NGR | 11.5 ± 3.5 | 12.0 ± 2.8 | 12.0 ± 2.7 | 13.0 ± 4.9 | 13.5 ± 4.5 | 12.5 ± 3.8 |
| DHA | 10.0 ± 3.7 | 11.5 ± 3.4 | 12.5 ± 2.3 | 14.5 ± 6.2 | 14.0 ± 4.8 | 14.0 ± 4.0 |
| NGF + DHA | 12.0 ± 3.4 | 12.5 ± 3.7 | 13.5 ± 3.9 | 15.5 ± 5.3 | 15.0 ± 5.4 | 14.5 ± 3.2 |

*Values are mean ± Standard Error (n = 6, for each treatment; n = 3, for the control)

The results of the tear break-up time measurements (Table 1) were approximately 25% smaller than previously published for rabbits, possibly because they were performed under anesthesia in order to allow an easier and more reliable result. See S. Barabino et al., “Tear film and ocular surface tests in animal models of dry eye: uses and limitations,” Exp. Eye Res., vol. 79, pp. 613-621 (2004). At 1 week postoperative, the tear break-up time measurement was 13 sec in the control and in the NGF-treated group, 14.5 sec in the DHA-treated group, and 15.5 sec in the NGF plus DHA-treated group (P=0.72). The values at 1 month were 12.5, 13.5, 14.0, and 15.0 sec for controls, NGF-, DHA- and NGF plus DHA-treated groups, respectively (P=0.78). These differences were not statistically significant.

At the first postoperative month, positive rose bengal staining was noted in 50% of control eyes and 33% of DHA-treated eyes. Only 16% of the eyes treated with NGF and no eyes treated with NGF plus DHA showed positive rose bengal staining. Similar results were seen at 15 and 45 days after surgery. However, no difference in the Schirmer's test at 15 days, 1 month, or 6 weeks after PRK was found between the group of eyes that had positive rose bengal staining and the group that did not show staining, regardless of the treatment. (Table 2)

TABLE 2
Schirmer's Test in Rabbits with Positive or Negative Rose Bengal Staining

| | Rose Bengal | No Rose Bengal | P |
|---|---|---|---|
| Days After PRK | Staining Group (mm) | Staining Group (mm) | Value |
| 15 Days | 12.7 ± 4.2 | 13.2 ± 5.2 | 0.79 |
| 30 Days | 13.4 ± 5.1 | 13.7 ± 5.4 | 0.82 |
| 45 Days | 13.7 ± 4.8 | 13.8 ± 4.6 | 0.91 |

*Samples were grouped according to Rose Bengal Staining without consideration to the treatment.

There were no significant differences in the tear secretion tests between the four groups. However, none of the eyes treated with NGF plus DRA developed rose bengal staining 30 days after PRK as compared with 50% in the control group, 33% in the DHA-treated group, and 16% in the NGF group. These results indicated that acceleration of nerve regeneration appearred to be associated with improved epithelial cell integrity. However, no difference was found between the treatment and control groups with regards to Shirmer testing and tear break-up time. This is in agreement with a study with patients who underwent LASIK surgery in which no correlation between decrease in tear production using Schirmer's test and changes in corneal sensitivity was found. (Michaeli et al., 2004) These findings indicated that the punctate epithelial erosions and rose bengal staining that develop after PRK are not attributable to diminished tear production, but may be the result of a PRK-induced neurotrophic epitheliopathy caused by diminished neurotrophic factors released from the injured and partially regenerated nerve endings. The combination of DHA and NGF completely inhibited epithelial defects, and in fact, increased epithelial proliferation. The combination of DHA and NGF was a significantly better treatment for enhancing nerve regeneration in the corneal after PRK than treatment with only NGF.

### EXAMPLE 4

Tissure Preparation, Staining and Analysis

Tissue Preparation

Rabbits were humanely euthanized at 8 weeks post-PRK surgery using an intravenous overdose of pentobarbital. The treated eyes were immediately enucleated, and the entire corneas excised and fixed in neutral formalin (10%) for 24 h. The corneas were removed, bisected, and embedded in optimal cutting temperature (OCT) medium (Miles, Inc.; Elkhorn, Ind.). Six μm cryostat sections were prepared, air-dried, and stored at −80° C. until further use. Each section was evaluated with hematoxylin and eosin (H&E) stain and by immuno-histochemical analysis.

Immunostaining

To identify epithelial and sub-basal regenerating nerve bundle endings after PRK, monoclonal antibodies for class III β-tubulin, calcitornin gene-related peptide (CGRP), and substance P (SP) were used. Tissue sections were incubated with mouse anti-class III β-tubulin antibody (Covance Research Products, Inc.; Berkley, Calif.) at a concentration of 1:500 for 1 hr, followed by incubating with a secondary antibody, fluorescein-conjugated horse anti-mouse (1:500) (Vector Labs, Inc., Burlingame, Calif.) for 45 min at room temperature. Tissues were also incubated with chicken anti-CGRP monoclonal antibody (1:500) (Chemicon International; Temecula, Calif.) at room temperature for 1 hr, followed by 1 hr with the secondary antibody, fluorescein-conjugated goat anti-chicken (1:1000) (Rockland, Gilbertsville, Pa.). Incubation was also conducted with guinea pig anti-SP monoclonal antibody (1:300) (Chemicon International) at room temperature for 90 min followed by the secondary antibody, fluorescein-conjugated goat anti-guinea pig (1:1000) (Santa Cruz Biotechnology Inc, Santa Cruz, Calif.) for 1 hr at room temperature.

Immunofluorescence with a monoclonal anti-chondroitin sulfate clone CS-56 (Sigma Chemical Co.) was performed as previously described in S. Esquenazi et al., “Prevention of experimental diffuse lamellar keratitis using a novel platelet-activating factor receptor antagonist,” J. Cataract Refract. Surg., vol. 30, pp. 884-891 (2004).

To stain for rabbit corneal myofibroblasts (RCM), tissue sections were incubated with (1:300) monoclonal mouse anti-alpha smooth muscle (αSMA) (Sigma Chemical Co.) for 2 hr at room temperature, followed by incubation with the secondary antibody fluorescein conjugated goat anti-mouse IgG (Vector Labs Inc) for 1 hr at room temperature.

To study proliferating cells in the epithelium and anterior stroma, tissue sections were incubated with 1:100 dilution of monoclonal mouse anti-human Ki-67 primary antibody (Sigma Chemical Co.) for 2 hr. To observe anterior stromal scarring and haze formation, tissue sections were incubated with 1:300 monoclonal mouse anti-collagen III antibody (Sigma Chemical Co.) for 1 hr. Both stains were followed by incubation with the secondary antibody fluorescein conjugated horse anti-mouse IgG (Vector Labs Inc).

In all tissue sections, cover slips were mounted with Vectashiel mounting medium H: 1000 (Vector Labs Inc). For nuclear counterstaining, DAPI solution was used according to the manufacturer's recommendations. Photographs were taken with a Nikon Eclipse TE 200 fluorescence microscope equipped with a Nikon DXM 1200 digital camera (Nikon Inc, Melville, N.Y.).

Tissue Area and Cell Number Measurements

Photographs of the tissue sections were acquired using MetaVue version 5.0r3 (Universal Imaging Corp.; Downingtown, Pa.) and saved as a TIFF file. (Data not shown) The tubulin III-, CGRP- and SP-positive tissue nerve areas and the percentage of Ki-67 cells were calculated with respect to the total area using the image analysis program Image Pro Plus 4.5 (Media Cybernetics Inc., Silver Spring, Md.). Sub-basal and epithelial nerve areas were measured in all groups eight weeks after PRK using anti-class III β tubulin, CGRP and SP monoclonal antibodies. The ratios of antibody-positive sub-basal nerve area to the stromal area, and of antibody-positive epithelial nerve area to the total epithelial area of the tissue were determined.

Results of Tissue Staining

Two months (8 weeks) after PRK, Ki-67 positive cells were observed predominantly in the basal epithelium of all eyes (picture not shown). Eyes treated with NGF and NGF plus DHA showed more intense staining compared with the DHA-treated or control groups. When the percentage of Ki-67 positive epithelial cells was determined, tissue that was treated with NGF+DHA showed 19% positive Ki-67 cells as compared with 15%, 6% and 5% in the NGF, DHA and control groups, respectively. (n=6 for each treatment; n=3 for control). Significant increases in proliferative cells over the control were found with both NGF treatment (p<0.001) and with NGF plus DHA treatment (p<0.001).

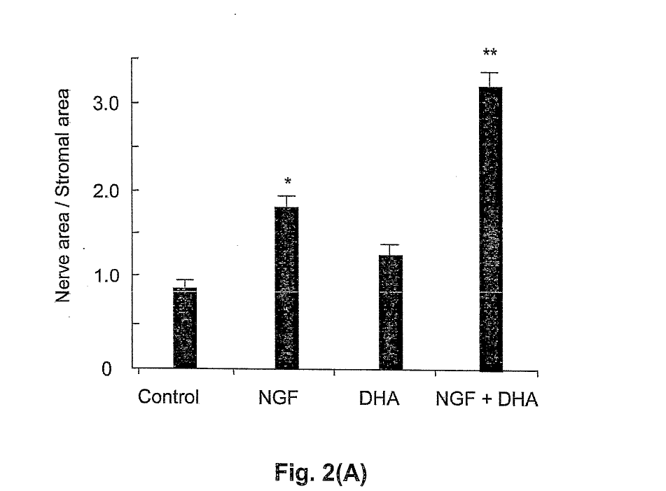

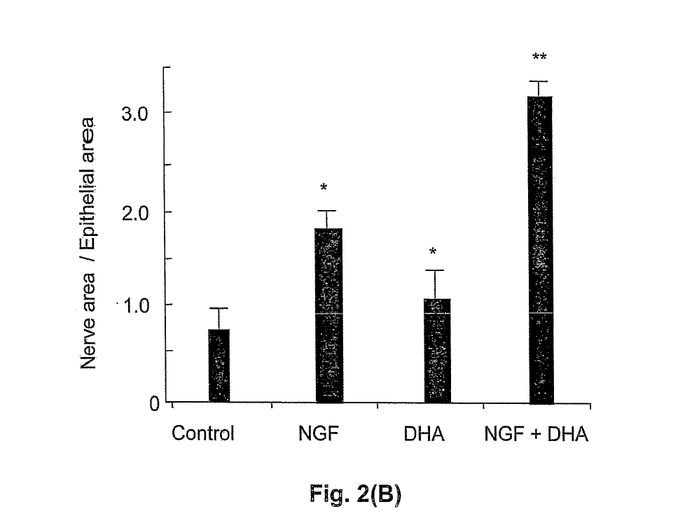

The effects of NGF and DHA on tubulin-positive sub-basal and epithelial nerve bundles are shown in FIGS. 2A and 2B. Increase in tubulin staining in the sub-basal and epithelial nerve bundles was observed in the NGF- and NGF plus DHA-treated groups compared with controls and DHA-treated groups. (Pictures not shown) The nerve area was calculated with respect to the total tissue area. Each bar represents a mean±standard error (n=6 in the treated groups; n=3 in the control group). A significant difference from the control is indicated as * (p<0.05). A highly significant increase over the group treated with only NGF is indicated by ** (p<0.001). As shown in FIG. 2A, the average ratio of sub-basal tubulin-positive nerve bundle area to total area in controls was 0.85. Treatments with only NGF and only DHA produced ratios of about 1.8 and 1.2, respectively, both showing a significant increase in nerve bundle area over the control (p<0.05). The combination of NGF and DHA produced a significant, synergistic increase over treatment with NGF alone in the sub-basal tubulin positive nerve area with an average ratio of 3.1 (p<0.001). In the epithelial layer, the ratio of the tubulin positive nerve bundle area to the total epithelial area in the controls was a mean of 0.78 (FIG. 2B). Treatment with only NGF increased the ratio significantly (p<0.05), but DHA alone had no significant effect. Treatment with the combination of NGF and DHA produced the greatest effect, showing an average ratio of cover 3.0, a significant increase (p<0.001)

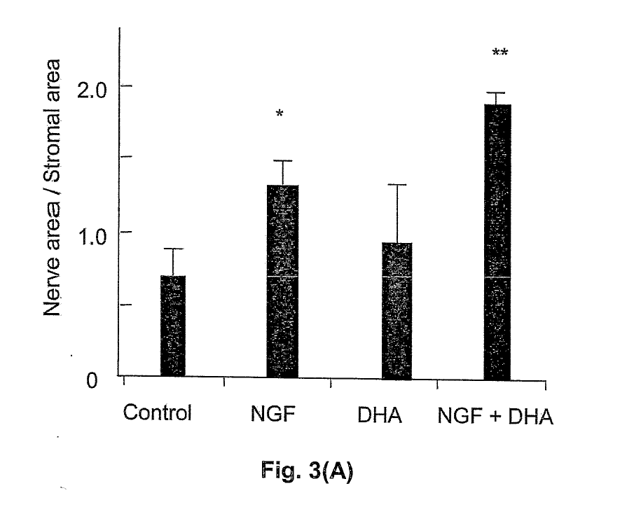

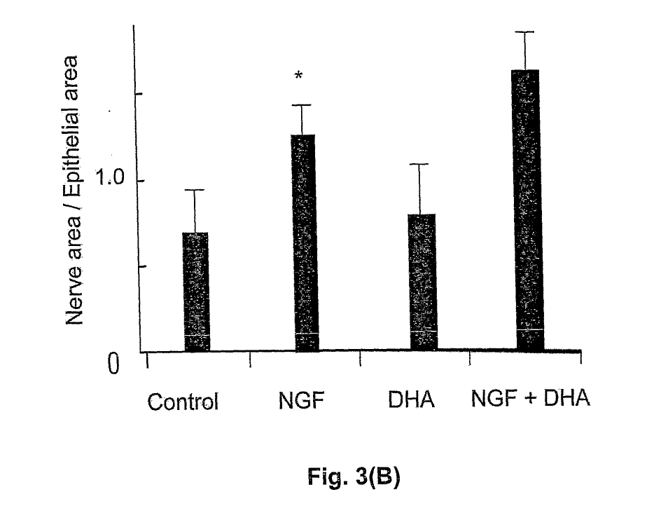

Increased staining with CGRP antibody of sub-basal and epithelial nerve bundle areas was seen in the presence of NGF and DHA. (FIGS. 3A and 3B; pictures not shown) In FIGS. 3A and 3B, the nerves were stained with CGRP immnofluourescent stain, while the nuclei of epithelial and stromal cells were counterstained with DAPI, as discussed above in Example 1. Each bar represents a mean±standard error (n=6 in the treated groups; n=3 in the control group). A significant difference from the control is indicated as * (p<0.05). A highly significant increase from the group treated with only NGF is indicated by ** (p<0.001). Eight weeks after PRK, the controls showed an average epithelial and sub-basal CGRP-positive nerve area relative to the entire corneal tissue as 0.68 and 0.62 mm²/mm² (nerve/corneal tissue), respectively. (FIGS. 3A and 3B). A statistically significant increase in the ratios of CGRP-positive sub-basal and epithelial nerve bundles to the respective total area were noted in the group treated only with NGF compared to the controls (p<0.05); however, no statistically significant difference was observed between the group treated only with DHA and the control group. The ratio of the nerve area in both the epithelial and stromal areas of the group treated with NGF plus DHA was significantly increased with respect to the group treated only with NGF (p<0.001).

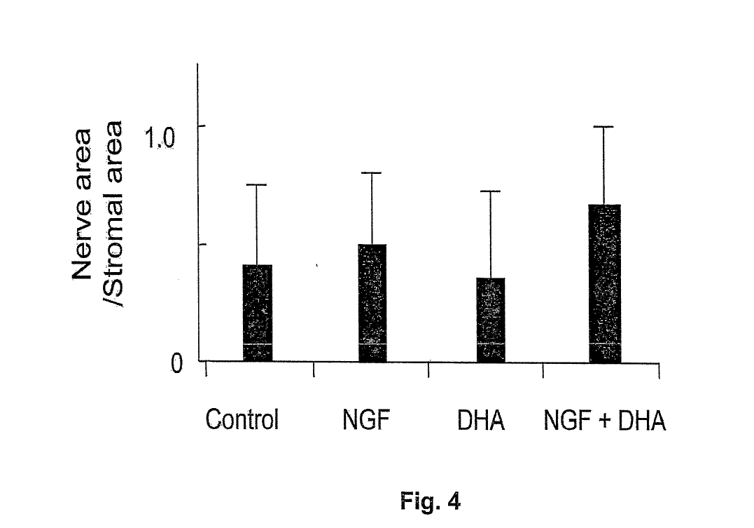

Nerve staining with Substance P was much lower than with tubulin or CGRP. Two months after PRK, the nerve bundle area without treatment was 0.38, and no significant differences were observed among any of tihe groups (FIG. 4).

Collagen III expression and chondroitin sulfate staining was observed in the anterior stroma 8 weeks after PRK, but no significant differences were observed among any of the groups (data not shown). In contrast, no α-SMA staining was observed in any group.

Thus a higher percentage of Ki-67-positive cells, a marker of cell proliferation, was observed in the DHA plus NGF and NGF-treated groups compared with DHA alone or controls. Eight weeks after PRK, tubulin-positive and CGRP-positive epithelial and sub-basal nerve bundle areas were significantly higher in the DHA plus NGF group compared to controls and to either NGF or DHA alone. 7Thus DHA alone showed no increase in nerve density in the sub-basal and epithelial areas, as compared to controls. However, the combination of DHA and NGF resulted in a two-fold increase in positive nerve tissue stained with tubulin and CGRP even as compared to the NGF group. The number of identifiable SP-positive neurons was very low and no differences were seen among all groups. A previous study has reported that about 58% of corneal neurons are CGRP-positive while only 20% are SP-positive. (C. Belmonte et al., 2004).

The above results indicated that NGF plus DHA treatment after PRK in rabbits was associated with increased corneal nerve surface area, increased epithelial proliferation, and decreased rose bengal staining as compared with NGF, DHA, or vehicle control alone. The combination of NGF plus DHA thus yield faster nerve recovery after PRK and has therapeutic importance in the treatment of post-PRK dry eye and other neurotrophic keratopathies.

As used in the specification and claims, an “effective amount” of of the NGF plus DHA-albumin complex that is sufficient to increase the degree of re-innervation after PRK or LASIK or other disruption to the cornea to a clinically significant degree. Significance for this purpose is determined as the P<0.5 level, or by such other measure of statistical significance as is commonly used in the art for a particular type of experimental determination. The dosage ranges for the administration of NGF plus DHA-albumin are those that produce the desired effect. Generally, the dosage will vary with the age and condition of the patient. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the increase in corneal nerve area by methods well known to those in the field and by methods taught by this Specification. Moreover, the NGF plus DHA can be applied in pharmaceutically acceptable carriers known in the art. The application is preferably topical.

Controlled delivery may be achieved by admixing the active ingredient with appropriate macromolecules, for example, polyesters, polyamino acids, polyvinyl pyrrolidione, ethylenevinylacetate, methylcellulose, carboxymethylcellulose, prolamine sulfate, or lactide/glycolide copolymers. The rate of release of NGF plus DRA may be controlled by altering the concentration of the macromolecule.

Another method for controlling the duration of action comprises incorporating the DHA-albumin complex into particles of a polymeric substance such as a polyester, peptide, hydrogel, polylactide/glycolide copolymer, or ethylenevinylacetate copolymers. In addition, the DHA-complex can be administered using a collagen shield or contact lens that is somewhat absorbent of the complex, e.g., Soft Shield Collagen Shield, 72-hour (Oasis Medical Inc., Glendora, Calif.), hilafilcon B soft 2-week contact lens (Bausch & Lomb, Rochester, N.Y.), and Night and Day soft contact lenses (Ciba Vision, Duluth, Ga.). The shield or lens can be made of any hydrophilic transparent polymer, such as poly-hydroxyethylmethacrylate hydrogel, ethoxy ethyl methacrylate hydrogel, methacrylic acid, n-vinylpyrolidinone, siloxane hydrogel, polydimethylsiloxane polyols, perfluoropolyethers, dimethylacrylamide, methyl methacrylate, and fluorosiloxane hydrogel, as discussed in P. C. Nicolson et al., “Soft contact lens polymers: an evolution,” Biomaterials, vol. 22, pp. 3273-3283 (2001).

The present invention provides a method of treating or attenuating the symptoms of dry eye or other neurotrophic keratopathies resulting from some disruption to the corneal nerve supply, comprising topically administering to a patient who has an injured cornea (e.g., one who has undergone PRK or LASIK) an effective amount of NGF plus DHA-albumin complex. The term “attenuate” refers to a decrease or lessening of the symptoms or signs of such nerve problems.

The complete disclosures of all references cited in this specification are hereby incorporated by reference. In the event of an otherwise irreconcilable conflict, however, the present specification shall control.

What is claimed:

1. A method to enhance nerve re-generation in an injured cornea, said method comprising topically administering to the injured cornea an effective amount of a combination of nerve growth factor and docosahexaenoic acid.

2. A method as in claim 1, wherein said cornea is injured by a cause selected from the group comprising trauma, photorefractive keratectomy (PRK), laser in situ keratomileusis (LASIK), chemical burn, congenital corneal neuropathy, and acquired corneal neuropathy.

3. A method as in claim 1, wherein said cornea is injured during laser in situ keratomileusis (LASIK).

4. A method as in claim 1, wherein said cornea is injured during by photorefractive keratectomy (PRK).

5. A method to alleviate symptoms of dry eye from an injury to a cornea, said method comprising topically administering to the injured cornea an effective amount of a combination of nerve growth factor and docosahexaenoic acid.

6. A method as in claim 5, wherein said cornea is injured by a cause selected from the group comprising trauma, photorefractive keratectomy (PRK), laser in situ keratomileusis (LASIK), chemical burn, congenital corneal neuropathy, and acquired corneal neuropathy.

7. A method as in claim 5, wherein said cornea is injured during laser in situ keratomileusis (LASIK).

8. A method as in claim 5, wherein said cornea is injured during by photorefractive keratectomy (PRK).

9. A method to alleviate symptoms of neurotrophic keratopathy from an injury to a cornea, said method comprising topically administering to the injured cornea an effective amount of a combination of nerve growth factor and docosahexaenoic acid.

10. A method as in claim 9, wherein said cornea is injured by a cause selected from the group comprising trauma, photorefractive keratectomy (PRK), laser in situ keratomileusis (LASIK), chemical burn, congenital corneal neuropathy, and acquired corneal neuropathy.

11. A method as in claim 9, wherein said cornea is injured during laser in situ keratomileusis (LASIK).

12. A method as in claim 9, wherein said cornea is injured during by photorefractive keratectomy (PRK).

13. A composition comprising a mixture of an effective amount of nerve growth factor, an effective amount of docosahexaenoic acid, and a pharmaceutically acceptable carrier; wherein said composition is sterile; and wherein said composition is suitable for topical application to a human cornea in vivo.

14. A composition as in claim 13, wherein said docosahexaenoic acid is bound to albumin.

15. An article of manufacture comprising a sterile covering adapted to protect an injured human cornea in vivo; wherein said covering comprises an effective amount of a composition as recited in claim 13; and wherein said article is adapted to release said composition over time when in contact with a cornea in vivo.

16. A covering as in claim 15, wherein said docosahexaenoic acid is bound to albumin.

17. An article of manufacture as recited in claim 15, wherein said covering comprises collagen.

18. An article of manufacture as recited in claim 15, wherein said covering comprises a transparent polymer selected from the group consisting of poly-hydroxyethylmethacrylate hydrogel, ethoxy ethyl methacrylate hydrogel, methacrylic acid, n-vinylpyrolidinone, siloxane hydrogel, polydimethylsiloxane polyols, perfluoropolyethers, dimethylacrylamide, methyl methacrylate, and fluorosiloxane hydrogel.

19. An article of manufacture as recited in claim 15, wherein said covering additionally comprises a macromolecule selected from the group consisting of polyesters, polyamino acids, polyvinyl pyrrolidone, ethylenevinylacetate, methylcellulose, carboxymethylcellulose, prolamine sulfate, and lactide/glycolide copolymers; wherein said macromolecule will alter the rate of release of said composition when said article is in contact with a cornea in vivo, as compared with the rate of release from an otherwise identical article of manufacture lacking said macromolecule.
